(19) United States
(12) Patent Application Publication
(10) Pub. No.: US 2012/0288540 A1
(43) Pub. Date: Nov. 15, 2012

(54) NIOSOMES, FREEZE-DRIED POWDER THEREOF AND THEIR USE IN TREATMENT

(75) Inventors: Maria Carafa, Rome (IT); Franco Alhaique, Rome (IT); Carlotta Marianecci, Rome (IT); Donatella Paolino, Catanzaro (IT); Massimo Fresta, Catanzaro (IT)

(73) Assignees: UNIVERSITÀ DEGLI STUDI "MAGNA GRAECIA" CATANZARO, Catanzaro (IT); UNIVERSITÀ DEGLI STUDI DI ROMA "LA SAPIENZA", Rome (IT)

(21) Appl. No.: 13/519,200

(22) PCT Filed: Jan. 5, 2011
(86) PCT No.: PCT/IB2011/050041

§ 371 Date: Jun. 26, 2012

(30) Foreign Application Priority Data
Jan. 7, 2010 (IT) RM2010A000002

(51) Int. Cl. A61K 9/00; A61P 11/00; A61K 47/28

(52) U.S. Cl. 424/400; 514/772; 514/777

(57) ABSTRACT

The present invention relates to a vesicular structure formed of a mixture of a non-ionic surfactant and cholesterol, characterised in that it is freeze-dried in the presence of a cryoprotectant, to a method for the preparation and to the use thereof.

### BACKGROUND OF THE INVENTION

The present invention relates to niosomes, in particular in the form of freeze-dried powder and their use in treatment. The present invention is particularly advantageous in the treatment of respiratory pathologies.

### PRIOR ART

COPD is a chronic and progressive disease of the airways which manifests itself in bronchial obstruction, which is rarely reversible, sustained by an inflammatory process of the airways and of the pulmonary parenchyma in response to various environmental triggers in predisposed subjects.

Clinically, it manifests itself in wheezing, difficulty in breathing and cough, above all at night time and in the morning, and by an increase in secretions.

Current first-choice treatment provides the use of inhaled corticosteroids which make it possible to control the symptoms, improve pulmonary function and bronchial hyperactivity, and modify bronchial inflammation and its consequences.

Beclomethasone dipropionate and other drugs of steroidal structure are widely used via inhalation as a result of their proven efficacy, however there is currently no uniformity between clinical results due to different variables which come into play for each inhalation pharmaceutical form with which these drugs are formulated. In fact, only a minimal amount of an aerosolised drug is deposited in the lower airways, generally between 2 and 10%, a large part of the remaining drug being ingested and absorbed by the gastrointestinal system causing systemic side-effects. Furthermore, the percentage of the drug deposited at pulmonary level has only little or no chance of diffusing through the bronchial mucosa characterised by an increased mucous layer and therefore has a purely superficial therapeutic effect.

Furthermore, following systemic absorption, all the glucocorticoids exhibit side-effects even severe when administered over long periods and at high doses, such as: reduction or suppression of the adrenal function, effects on bone metabolism which could increase the risk of osteoporosis, depression of the immune system with potential overlapping infections and an increase in susceptibility thereto, etc.

Therefore, it is necessary to develop new ‘carrier’ systems able to transport drugs directly to the site of action.

For many years studies have been carried out on the optimisation of particular non-phospolipid vesicular structures for drug delivery (Azeem, A., Anwer, K., Talegaonkar, S. J. Of Drug Targeting (2009), 17, 671-689; Schatzlein, A. G. Drug Targeting and Delivery (2000), 11, 153-184). More recently, a study aimed at the optimisation of such systems for glucocorticoid lung targeting has been initiated.

As already mentioned, such drugs are used in the treatment of respiratory pathologies such as asthma and chronic obstructive pulmonary disease (COPD), for which it is essential that an effective dose of the drug reaches the receptor localised in the cytoplasm of bronchial fibroblasts which is not easily accessible using conventional administration methods, in particular in the presence of obstructive secretions.

Until now, no other study on the use of non-phospholipid vesicular structures for lung targeting has been reported in the literature, particularly with regard to the possibility of obtaining such systems using a powder.

### DESCRIPTION OF THE INVENTION

The present invention describes the application of nanotechnology for drug delivery, in particular beclomethasone dipropionate, within the respiratory system. In particular, the inventors propose niosomes, surfactant-based vesicular structures, as carrier systems. Niosomes are vesicular structures formed of a bilayer of non-ionic surfactant molecules which contains an aqueous nucleus. Such structures, which are similar in formation and structure to liposomes, well-known vesicles formed of phospholipid molecules, can carry molecules having different features: hydrophilic molecules which can be entrapped in the aqueous nucleus and hydrophobic substances which can be connected to the bilayer.

These vesicles containing the drug have proven to be particularly stable over time and exhibit increased biocompatibility (MTT test as reported in the proposal). Niosomes have also demonstrated the ability to interact with cellular structures (human bronchial fibroblasts) and to increase the entry of the drug into the cytoplasmic compartment of human bronchial fibroblasts compared to the drug administered as aqueous suspension.

A further positive aspect which can be evidenced in the proposed invention lies in the freeze-drying of the proposed niosomal suspension, thus making it possible to eliminate the drawback of the loss of active ingredient from the niosomal vesicles during the life cycle of the medicinal product as well as the possible aggregation of said vesicles (that is to say from before administration).

The inhalation powder is more stable and can be dosed more easily than an aqueous suspension since the patient breathes a dose of the drug directly using the powder dispenser nozzle.

The possibility of having a formulation in solid phase rather than in liquid phase therefore affords an enormous advantage since it is possible to envisage production of a commercial product in the form of pre-dosed powder inhalers which are very practical and convenient in chronic pathologies such as COPD.

There are many innovative features of the invention, but these can be summarised in two key points: delivery of the drug, for example beclomethasone diproprionate via niosomes at pulmonary level and freeze-drying of the niosomal vesicles intended for pulmonary administration. Both of these features result in substantial and significant improvements compared to current treatments since no product having features similar to those proposed are currently commercially available.

The proposed vesicular structures therefore are of great interest and can be applied in the pharmaceutical field and, in particular, for drug delivery at bronchial level. Once patent protection has been obtained, it will be possible to supplement testing with clinical data. The proposed embodiment of the invention will surely spark great scientific and commercial interest from pharmaceutical companies operating within the sector of drug delivery within the respiratory system since the proposed invention is rather innovative and could constitute a real advance on the prior art in the relevant sector.

Vesicular structures formed of polyoxyethylene sorbitan monolaurate (Tween 20), a commercial surfactant, and cholesterol have demonstrated the capacity to promote the diffusion of a lipophilic drug, beclomethasone dipropionate (BDP), through the mucous when compared to commercial formulations (“Non phospholipid vesicles for pulmonary glucocorticoid delivery” C. Terzano, L. Allegra, F. Alhaique, C. Marianecci, M. Carafa Eur. J. Pharm. Biopharm. 2005, 59, 57-62).

During the course of a new study, the authors observed that such structures exhibit an increased capacity for interaction with human bronchial fibroblasts, wherein the BDP receptor can be found, which is fundamental for assessing an increased selectivity of drug delivery.

The studies regarding interaction and intracellular release were carried out using confocal microscopic techniques (CLSM).

It is possible to obtain carrier systems having characteristics of increased biocompatibility, (MTT test), by using vesicles formed of a non-ionic surfactant mixture, for example Tween 20 and cholesterol, with a diameter in the range of 170 nm to 200 nm and with a superficial charge potential) of approximately −40 mV.

Such vesicles exhibit increased stability over time (according to data from DLS and Turbiscan).

Furthermore, in order to resolve the aforementioned drawbacks of stability compared to aqueous suspensions and to increase the patient compliance, a freeze-drying technique has been set up, able to keep the vesicular structures intact (as evaluated by electron microscopy after freeze-fracture), thus avoiding the loss of drug.

The innovative features of the invention are linked to the possibility of obtaining a stable and easy-to-dose freeze-dried powder of such carrier/BDP.

Freeze-drying of vesicular structures which encapsulate drugs increases the physical and chemical stability of the formulations, since it drastically reduces the problem of aggregation of the vesicles and that of loss of encapsulated drug (leakage). It is therefore an object of the invention a vesicular structure formed of a mixture of a non-ionic surfactant and cholesterol, characterised in that it is freeze-dried in the presence of a cryoprotectant. Preferably the non-ionic surfactant is Tween 20. More preferably the non-ionic surfactant/cholesterol mass ratio is 1:1. In a preferred embodiment, the non-ionic surfactant/cryoprotectant mass ratio is between 1:0.5 and 1:2.

In one embodiment the cryoprotectant is saccharose, lactose, mannitol or trehalose. Preferably the vesicular structure of the invention has a diameter between 170 nm and 200 nm. Also preferably the vesicular structure of the invention has a zeta potential of approximately −40 mV.

In a preferred embodiment the vesicular structure of the invention comprises a pharmacologically or biologically therapeutic agent.

It is an object of the invention the vesicular structure of the invention for medical use; preferably for use for treatment of a respiratory pathology. More preferably the vesicular structure of the invention is administered via aerosol.

It is an object of the invention the use of the vesicular structure of the invention to encapsulate a pharmacologically or biologically therapeutic agent.

It is an object of the invention a method for the preparation of the vesicular structure of the invention, comprising:

a) solubilising a non-ionic surfactant and cholesterol in order to obtain a vesicular structure;

b) freeze-drying said vesicular structure in the presence of a cryoprotectant.

The cryoprotectant is preferably saccharose, lactose, mannitol or trehalose. Preferably the non-ionic surfactant/cryoprotectant mass ratio is between 1:0.5 and 1:2. Freeze-drying is preferably carried out at a pressure of 2 atm (1520 mm of Hg) and a temperature of −55° C.

The invention further relates to a pharmaceutical composition comprising the vesicular structure of the invention and suitable solvents and/or diluents.

The present invention will be described by non-limiting examples with reference to the following figures:

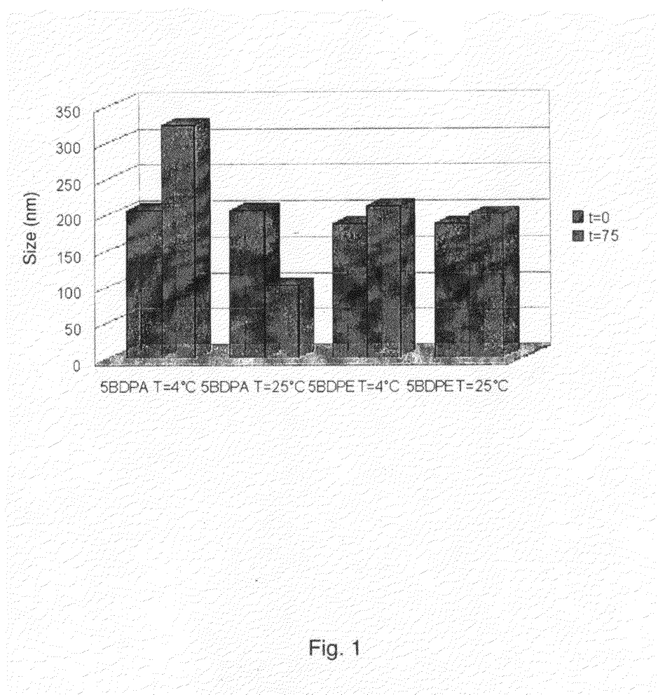

FIG. 1: measurements of stability over time, assessed as variation in sample size analysed at time zero and after 75 days.

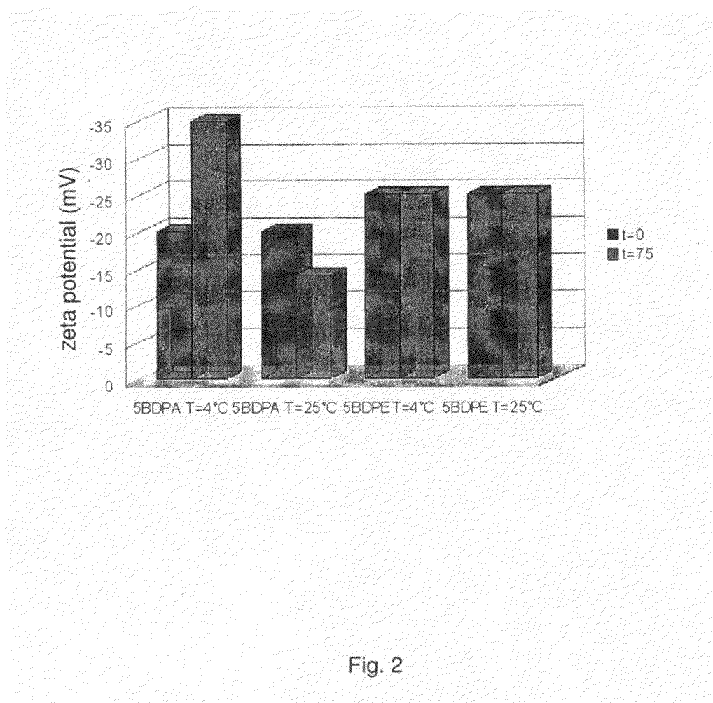

FIG. 2: measurements of stability over time, assessed as variation in zeta potential of the samples analysed at time zero and after 75 days.

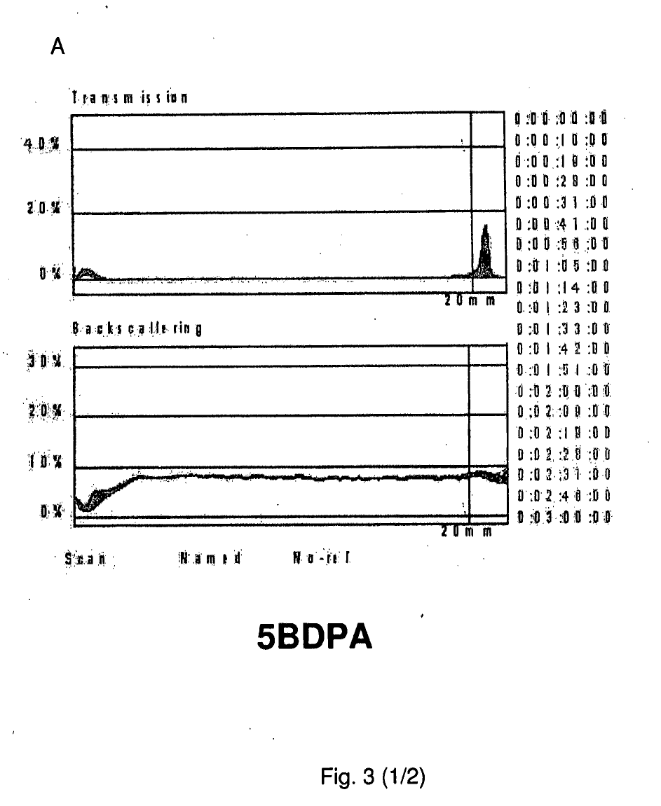

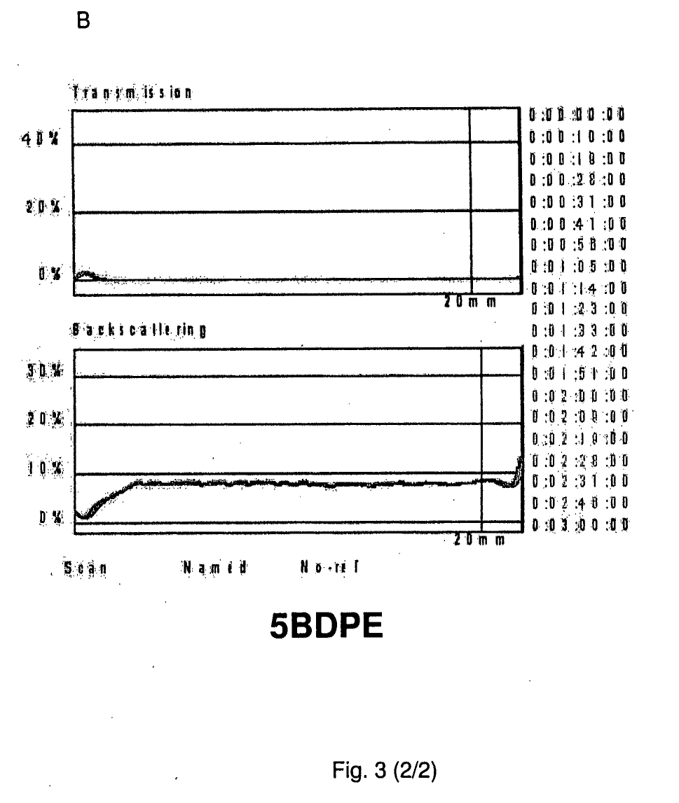

FIG. 3: transmission and back scattering spectra obtained for purified samples (A) of 5BDPA and (B) of 5BDPE using Turbiscanlab Expert apparatus.

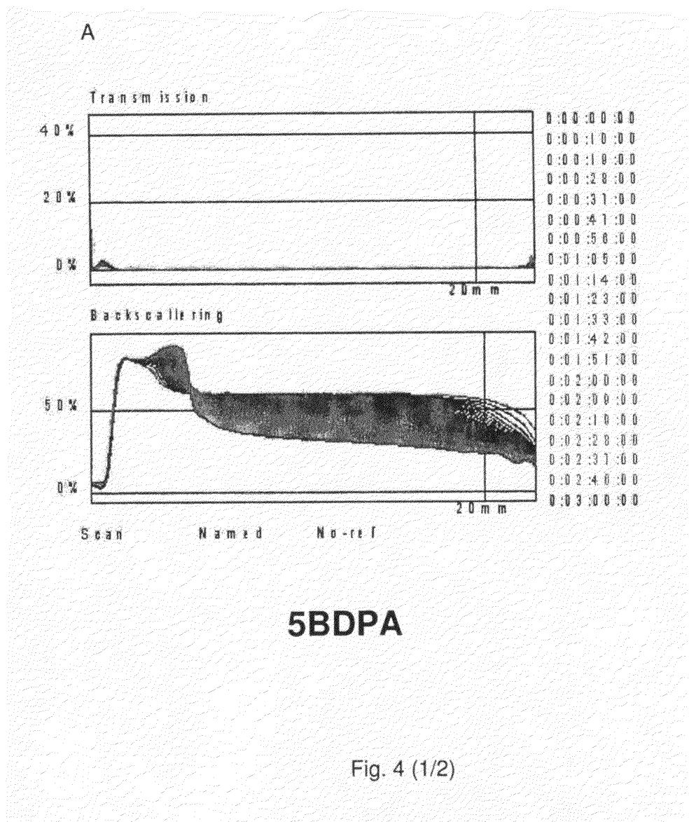

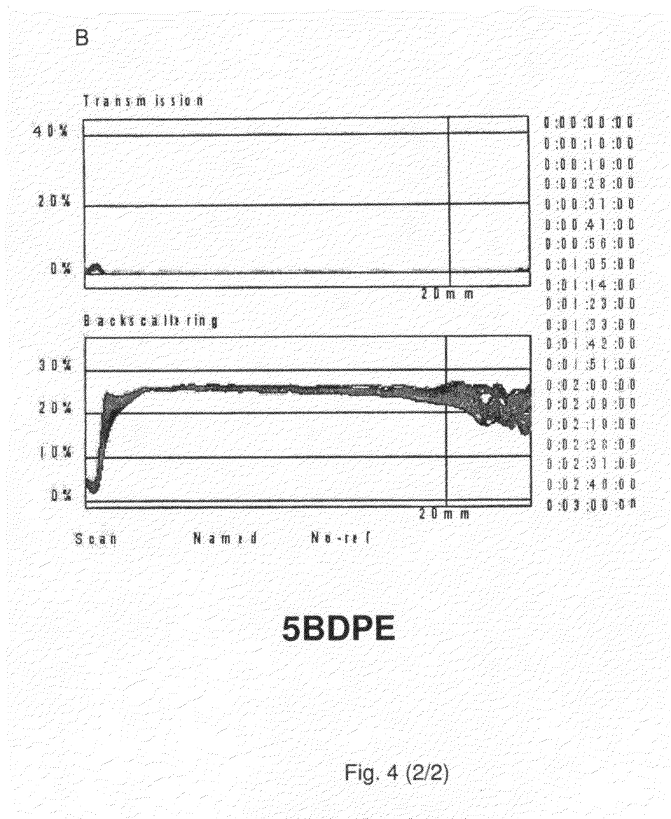

FIG. 4: transmission and back scattering spectra obtained for unpurified samples (A) of 5BDPA and (B) of 5BDPE using Turbiscanlab Expert apparatus.

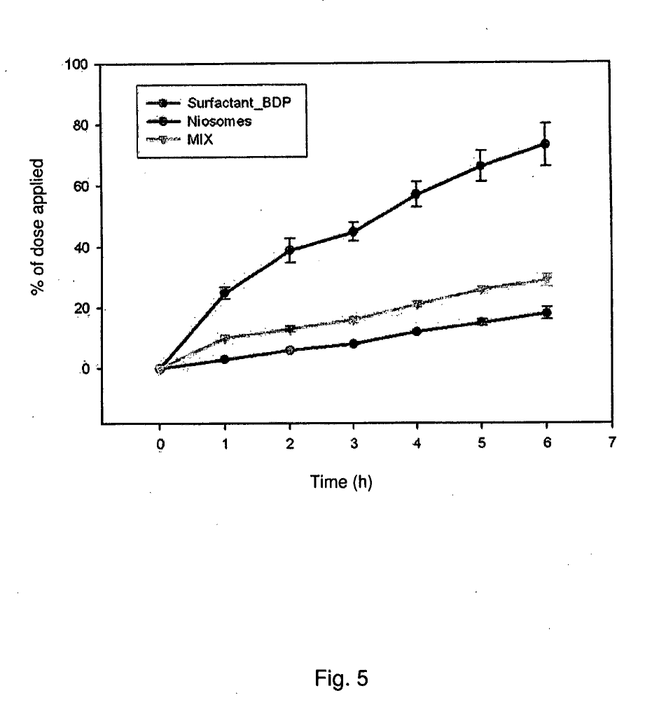

FIG. 5: % of BDP extracted in the bronchial fibroblasts compared to the dose applied for the samples as a function of time (in hours, h): 5BDPE (niosomes), BDP in the presence of Tween 20 not structured in vesicles (Surfactant_BDP) and a commercial mixture of BDP (Clenil®)(Mix).

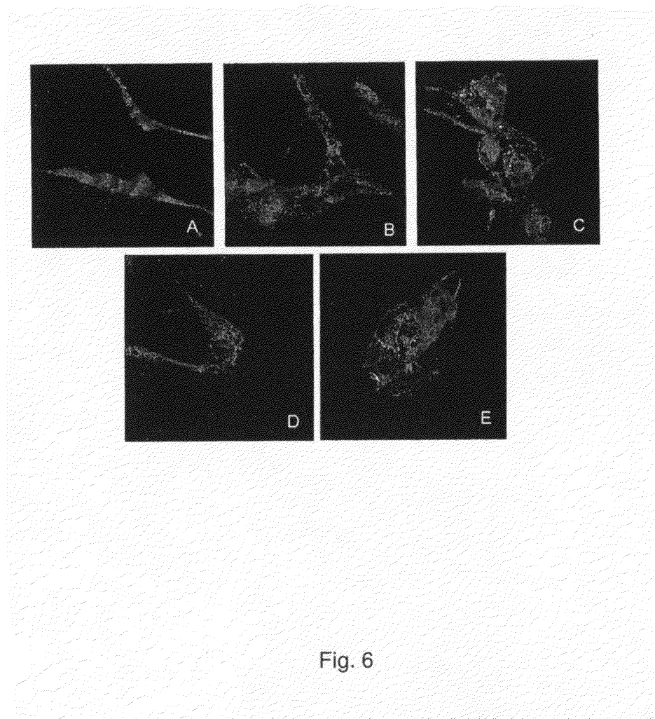

FIG. 6: micrographs obtained using confocal microscopy after interaction of the bronchial fibroblasts with niosomal vesicles containing 6-CF at different incubation time. A=1 h, B=4 h, C=6 h, D=12 h and E=24 h.

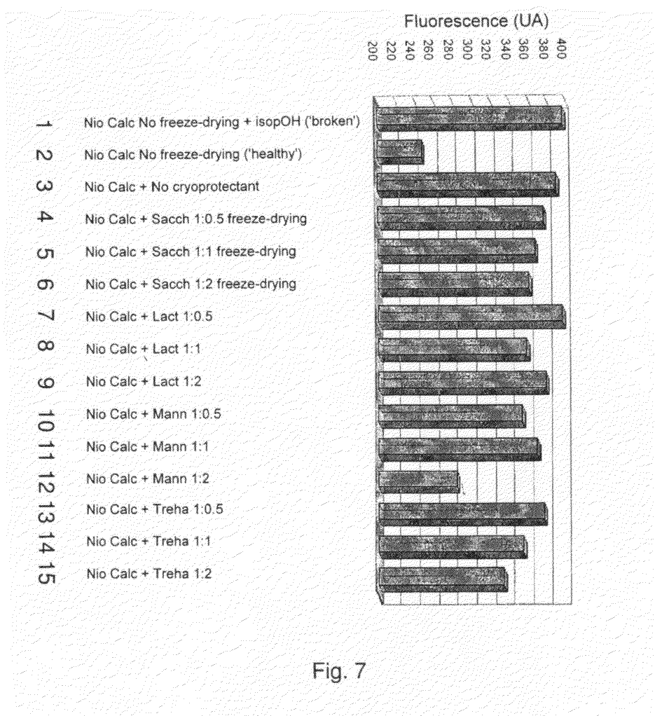

FIG. 7: fluorescence of the non-freeze-dried samples before and after breaking of the vesicular structures compared to that of the samples freeze-dryed in the absence or presence of cryoprotectants at different ratios: 1) complete, non-freeze-dried niosomes; 2) incomplete, non-freeze-dried niosomes; 3) niosomes freeze-dried in the absence of cryoprotectants; 4) niosomes freeze-dried in the presence of saccharose in a 1:0.5 ratio; 5) niosomes freeze-dried in the presence of saccharose in a 1:1 ratio; 6) niosomes freeze-dried in the presence of saccharose in a 1:2 ratio; 7) niosomes freeze-dried in the presence of lactose in a 1:0.5 ratio; 8) niosomes freeze-dried in the presence of lactose in a 1:1 ratio; 9) niosomes freeze-dried in the presence of lactose in a 1:2 ratio; 10) niosomes freeze-dried in the presence of mannitol in a 1:0.5 ratio; 12) niosomes freeze-dried in the presence of mannitol in a 1:2 ratio; 13) niosomes freeze-dried in the presence of trehalose in a 1:0.5 ratio; 14) niosomes freeze-dried in the presence of trehalose in a 1:1 ratio; 15) niosomes freeze-dried in the presence of trehalose in a 1:2 ratio.

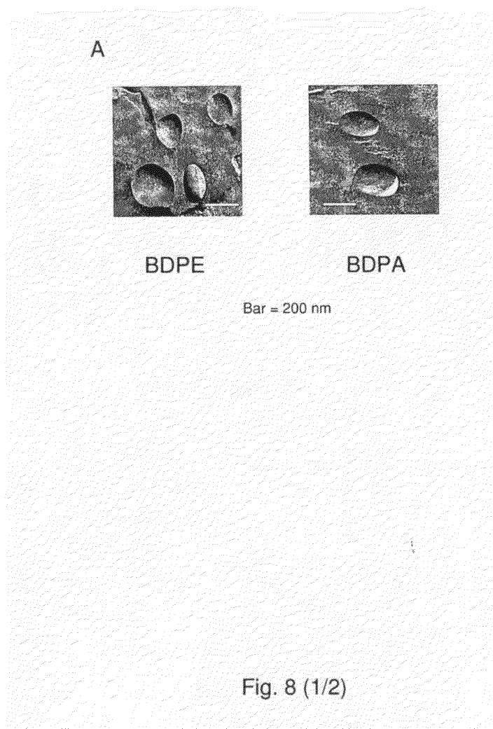

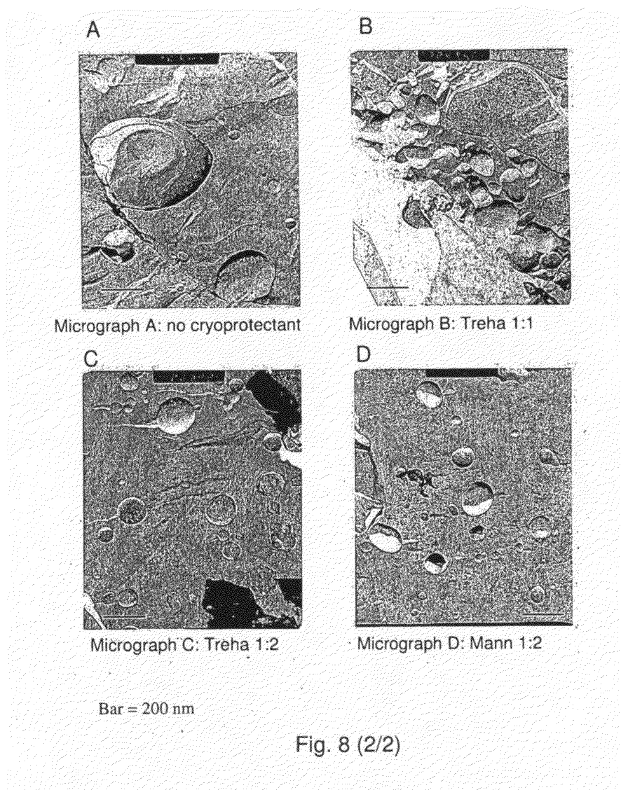

FIG. 8: images of vesicular structures (BDPA sample) obtained by electron microscopy before freeze-fracture (A′: non-freeze-dried sample) and after freeze-fracture in the absence of cryoprotectants (A), in the presence of trehalose in a 1:1 ratio (B), in the presence of trehalose in a 1:2 ratio (C) and in the presence of mannitol in a 1:2 ratio (D).

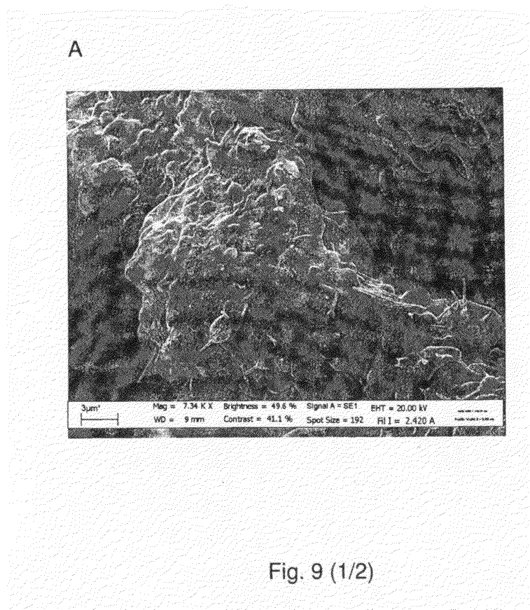

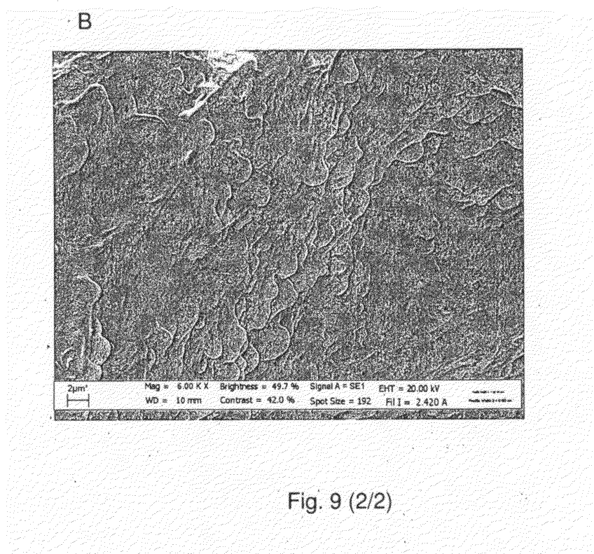

FIG. 9A SEM of BDPA freeze-dried in the absence of cryoprotectants

FIG. 9B SEM of BDPA freeze-dried in the absence of mannitol in a 1:2 ratio.

### MATERIALS AND METHODS

### Preparation of the Vesicular Structures

Polysorbate 20, cholesterol and beclomethasone diproprionate (BDP) were solubilised in organic solvent, then evaporated under vacuum until the formation of a film on the walls of a test tube. The film was rehydrated and then sonicated.

The formulations used are shown in Table 1.

TABLE 1
BDPA and BDPE formulations

| | Polysorbate 20 | | |
|---|---|---|---|
| Sample | (Tween 20) | Cholesterol | BDP |
| BDPA | 184 mg | 58 mg | 250 mg |
| BDPE | 184 mg | 58 mg | 2 mg |

The two drug concentrations selected in this study therefore are 50 mg/ml (BDPA) and 0.4 mg/ml (BDPE); the first formulation contains the maximum amount of drug that can be included in the vesicles; the second contains the same amount of drug as the commercial product Clenil for Aerosol®.

With the aim of eliminating the substances not included in the vesicles, after sonication the dispersion containing the niosomes is purified by gel filtration over a glass column (50×1.8 cm) packed with Sephadex G75 resin.

### Characterisation of the Vesicular Structures Size and ζ Potential

The dispersion containing the vesicles is diluted approximately 100 times in Hepes buffer at pH 7.4. The measurement is taken using a Malvern Nano. ZS90 (Malvern, UK) at 25° C., with a scattering angle of 90.0°.

The measurements of size and zeta potential were taken in order to characterise the samples (BDPA and BDPE) and in order to carry out stability studies. The stability of our formulations was assessed in terms of variation in size and zeta potential over a time interval of 75 days. The samples considered were stored at a temperature of 4° C. and 25° C.

### Studies of Stability Using Turbiscan Lab Expert

The stability of the niosomes was also assessed using Turbiscan Lab Expert. This apparatus is able to predict the long term stability of emulsions and colloidal systems. Turbiscan Lab operates based on the use of a pulsed light source in the near-infrared range (880 nm) and two synchronous detectors. The transmission detector receives the flow of light (T) transmitted through the analysed product and the backscattering detector measures the light (BS) refracted by the product)(135°. A computer combines the transmission and backscattering data to a fixed position in the cell, or every 40 μm, whilst it moves along the 55 cm of the cell.

The undiluted samples of the invention were analysed by acquisition of transmission and backscattering data every minute for a total duration of 3 hours, calculating physical parameters such as mean diameter of the particles and the volume fraction of the particles.

### Entrapment Efficiency

The percentage of drug present in the formulation contained in the vesicles can be determined by HPLC. All the analyses were carried out using a liquid chromatographic apparatus (Perkin-Elmer 250) with a Perkin-Elmer 235 photo-diode array detector, and a 50 μl (loop) Rheodyne injector connected to a computer; the column used is a Supelcogel ODP-50 (15 cm×4.00 mm I.D.); the mobile phase used is a 80/20 mixture of methanol/water; the BDP was revealed at 245 nm wavelength (λ) at a flow of 1.0 ml/min.

The entrapment efficiency (e.e.) was calculated using the following equation:

$$e . e . = \frac{100 \times mass of the drug included in the vesicles}{mass of the drug in the formulation}$$

### Aerodynamic Diameter (TOF ABS)

The TOF ABS is a technique able to determine the time of flight (TOF) elapsed between two laser beams arranged in transverse sequence to a flow of particles which emerge from a nozzle in a narrow flow. The path of the particles through the laser beam is examined by the apparatus by reading light scattering at 90°. A correlation system then makes it possible to carry out an analysis of 100,000 particles per second. Once the time of flight has been obtained, the mass of the particles, the volume and diameter can be deduced, by direct proportion, by inputting into the apparatus the data relating to the density of the solution under examination (API Aerosizer Mach2).

### Aerodynamic Diameter and Pulmonary Deposition

An aerosol is a suspension of particles (liquid or solid with a diameter between 1×10⁻³ and 100 μm) carried by a gas (generally air); this is therefore formed by a separate gaseous phase (air, oxygen) and by a particulate phase.

The distribution of the particles provided by various currently available apparatuses can be assessed statistically by studying two parameters: the mass median aerodynamic diameter (MMAD) and the geometric standard deviation (GSD), which are parameters used to assess the distribution of particles produced in the respiratory tract.

Study of Interaction with Human Bronchial Fibroblasts Cellular Uptake

The bronchial fibroblasts, incubated for 3 days at 37° C. (5% CO₂) using DMEM as a culture medium to which penicillin (100 UI/ml), streptomycin (100 μg/ml), an amphotericin solution B (1% v/v) and a solution of foetal bovine serum (FBS) were added were treated with 2 ml of trypsin solution (in order to remove adhesion of the fibroblasts to the glass), washed with 2 ml of a phosphate buffer and transferred to plastic centrifuge tubes. In order to obtain a final volume of 8 ml, 4 ml of the culture medium were added to the fibroblasts. The samples thus obtained were subjected to centrifugation using a Megafuge 1.0 (Heraeus Sepatech) at 1200 rpm for 10 minutes at ambient temperature. The cells thus obtained were separated from the supernatant and resuspended in 6 ml of culture medium in order to obtain 1×10⁶ cells/ml. The suspension of the cells was then diluted until a concentration of 3×10⁴ cells/ml was obtained and 1 ml of this suspension was introduced into various plastic culture dishes for subsequent in vitro tests, in particular in order to study the accumulation of the drug in fibroblasts after contact of such cells with the two niosomal suspensions containing BDP (BDPA and BDPE).

### Confocal Laser Scanning Microscopy (CLSM)

The interaction between the bronchial fibroblasts and the niosomal vesicles was studied by confocal laser scanning microscopy. The cells present for 24 hours in the culture vessels at a concentration of 3×10⁵ cells/ml were treated with a suspension of niosomes containing 6-carboxyfluorescein (6-CF) within the aqueous compartment, and incubated for different periods of time from 3 h to 24 h. The cells were then washed twice with 2 ml of a phosphate buffer at pH 7.4 and fixed for 3 minutes on the glas slide using 1 ml of ethanol solution (70% v/v). Fluorescence was then assessed by use of a Leika TCS PS2 MP confocal laser scanning microscope equipped with an argon-ion laser beam set to 496 nm excitation wavelength and 519 nm emission wavelength. A 4096×4096 pixel scanning resolution was used with a 75 mW Ar/Kr laser beam equipped with a filter for analysis of fluorescein. The data relating to the samples were acquired using ‘macro developer’ software with multidimensional acquisition series and direct access to the digital control systems.

### MTT Test (Vitality Test)

The MTT test is a colorimetric test which makes it possible to calculate the number of cells still exhibiting mitochondrial activity and therefore cell vitality. For each compound the IC₅₀ value is obtained by treatments at 14 hours and recovery at 48 hours, assessing the percentage of living cells by MTT test (a test of vitality based on a metabolic indicator—the soluble salt of tetrazole which, in living cells, is reduced from the mitochondrion due to the action of active dehydrogenase enzymes to form a water unsoluble purple crystal). The solubilised crystals are quantified using a colorimetric method at a wavelength of 570 nm (absorbance of the reduced colorant) with background correction at 690 nm.

### Freeze-Drying

Freeze-drying is a potential method for ensuring the long term stability of the vesicles and of the drug. In fact, freeze-drying makes it possible to remove the water from the system so as to prevent hydrolysis of the surfactant and makes it possible to obtain a powder which ensures chemical and physical stability. Furthermore, freeze-drying may lead to recovery of a powder which, as a result of its properties, could increase the efficiency of the productive processes. Freeze-drying of the suspension must be carried out so as to ensure that in the final powder the vesicular structures remain intact. Stabilisation of the vesicles by freeze-drying necessitates various steps: freezing of the vesicular suspension followed by removal of water by sublimation.

### Cryoprotection Tests

Fluorescence tests were used to first verify whether the niosomal vesicles were damaged during the process of freeze-drying. Then, in order to implement a cryoprotection method which would preserve the integrity of the vesicles, freeze-drying tests were carried out in the presence of various amounts of saccharose, lactose, mannitol and trehalose.

For this purpose niosomes containing calcein were used that had been prepared as detailed in the paragraph above.

Equivalent amounts by mass of cryoprotectant were added to known volumes of suspension, introduced into vacuum flasks, these amounts being doubled or halved relative to the mass of structured Tween 20 (41.5% of Tween 20 present in the initial formulation) contained in said volume. The various cryoprotectants tested were saccharose, lactose, mannitol and trehalose.

In order to determine the percentage of structured Tween 20 in the vesicle compared to the amount of surfactant in the initial formulation, a colorimetric method providing formation of a coloured complex which can be determined by UV spectroscopy was implemented.

TABLE 2
Ratio of Tween 20/cryoprotectant

| | Mannitol | Trehalose | Saccharose | Lactose |
|---|---|---|---|---|
| Ratio of | 1:0.5 | 1:0.5 | 1:0.5 | 1:0.5 |
| structured Tween | 1:1 | 1:1 | 1:1 | 1:1 |
| 20/cryoprotectant | 1:2 | 1:2 | 1:2 | 1:2 |

The lyophiliser used is an Edwards model operating at a pressure of 2 atm and at a temperature of −55° C. The samples thus obtained were resuspended in a volume of distilled water equal to the volume of suspension initially used and subjected to fluorimetric analyses: in particular the fluorescences of the various freeze-dried resuspensions thus obtained were compared with the fluorescences of the vesicles containing calcein not subjected to freeze-drying integer and destroyed with isopropanol.

### Preparation of Freeze-Dried Samples Containing BDP

The method of freeze-drying with cryoprotectant implemented by the experiments on vesicles containing calcein described above was used to freeze-dry vesicular suspensions containing BDP. The samples thus obtained were resuspended in a volume of water for HPLC equal to the volume of suspension used; HPLC analyses were carried out on these samples.

### Electron Microscopy After Freeze-Fracture

The freeze-fracture microscopy technique is an electron microscopy technique which is frequently used to study the internal organisation of membranes; it consists in rapidly freezing the ‘fresh’ biological sample and fracturing it using a cold blade; the plane of fracture is used as a basis for obtaining a complementary replica by deposition under vacuum of platinum-carbon; the final step consists in removing all biological material from the replica. At this point the replica can be analysed by electron microscopy. Generally, the sample is also pre-treated with glycerol so as to ensure cryoprotection.

This technique was used for characterisation of the vesicles containing BDP subjected to freeze-drying: the samples resuspended in distilled water were impregnated in 30% glycerol and then rapidly frozen in partially solidified Freon 22 then fractured in a freeze-fracture device (−105° C., 10⁻⁶ mmHg) and replicated by platinum-carbon deposition. The replicas were washed with distilled water and analysed using a Philips CM 10 electron microscope.

### Scanning Electron Microscopy (SEM)

SEM operates based on a bundle of electrons which is generated by an electron gun (cathode) arranged at the top of the column and is attracted toward the anode, condensed by collimating lenses and focussed on the sample through an objective lenses. The electron bundle hits the sample, producing inter alia secondary and retrodiffused electrons. These electrons are collected by a detector for secondary electrons and a detector for retrodiffuse electrons, are converted into electrical signals and amplified. These are converted into pixels and elaborated by a computer system.

SEM has a great depth of field which allows a vast part of the sample to be focused simultaneously. This can form an image which is a good three-dimensional representation of the surface of the sample since the apparatus has a resolution power of 3.5 nm and a maximum enlargement of 300,000 times.

Preparation of the sample is relatively simple since the apparatus used (LEO1450 VP) makes it possible to observe both conductive samples and insulating materials without any preparation.

In order to observe the surface morphology it is sufficient to fix, reversibly, the freeze-dried samples to a suitable metal support. Non-conductive materials are ‘metallised’, that is to say covered by a thin layer of gold or carbon, using a sputter/vaporiser available from the laboratory. The vesicles containing BDP (BDPA and BDPE) and freeze-dried with the various cryoprotectants were thus analysed by this method.

### RESULTS

The best formulation for carrying BDP is BDPA since it allows greater entrapment efficiency of the drug (77% of the initial BDP), whilst BDPE is the formulation in which BDP is present in concentrations equal to the formulation on the market which the authors took as a reference.

The experiments relating to size indicate that with an increase in the drug used in the formulation there is also an increase in the size of the vesicles (Table 3). This can be correlated to a distribution of the drug among the bilayer and the aqueous compartment of the vesicle. The presence of BDP in the formulation can influence the values of zeta potential which becomes ever closer to the value assumed by a suspension of BDP in a Hepes buffer. This effect may be caused by the steroid chemical structure of the drug which, in some ways, is similar to that of cholesterol and therefore enables its insertion within the bilayer.

TABLE 3
Sizes and zeta potential of the analysed samples,

| | | Zeta potential |
|---|---|---|
| Sample | Size (nm) | (mV) |
| BDPA | 205 ± 25 | −20 ± 0.3 |
| BDPE | 186 ± 24 | −25 ± 0.1 |
| Solution of 0.5% BDP p/v | | −30 ± 0.2 |

Since it was desired to assess the stability of the formulation of the invention over time, the authors measured size and zeta potential over a period of two and a half months, with storage of the vesicular suspensions at a temperature of 4° C. and 25° C.

It can be seen from FIGS. 1 and 2 that, in the case of the BDPE sample, there is no variation in zeta potential within the analysed period of time, either at a temperature of 25° C. or of 4° C. (FIG. 2), whilst a slight insignificant increase in size is observed at both temperatures (FIG. 1).

In the BDPA sample, in the case of storage at 4° C., there is an increase in size unexepectedly correlated with an increase in zeta potential. It is possible that the drug, present in an increased amount, migrates on the external surface of the vesicle, thus destabilising the bilayer and promoting the phenomena of fusion and coalescence between vesicles. At a temperature of 25° C. the stability of the product is very low; there is even a reduction in vesicular size and a reduction in zeta potential.

A further study of stability was carried out using the optical analyser Turbiscanlab Expert. This method was used to analyse the BDPA and BDPE suspensions before and after purification in a Sephadex G75 column. It is evident from the transmission and backscattering spectra that the purified samples do not exhibit aggregation, flocculation or phase segregation under the accelerated conditions to which they are subjected during analysis. This is evident from the spectra of the purified BDPA and BDPE illustrated in FIG. 3.

FIG. 4 shows the spectra of stability of unpurified BDPA and BDPE. It can be noted that there is a variation in backscattering (indicative of a reduction in stability) over time which is greater for the BDPA sample. It is possible that this reduction in stability is caused by the presence in suspension of drug not contained in the vesicles; in fact the effect is more evident in the case of the preparation containing more of the drug:

The authors also assessed the possibility of using these new vesicular dispersions in a conventional apparatus for aerosol therapy, the Pari Turboboy Jet Nebulizer. For this purpose the samples were characterised with regard to their aerodynamic diameter after nebulisation. Measurements of the MMAD (mass median aerodynamic diameter) and of the GSD (geometric standard deviation) were taken; the latter yielded values of 1.5, illustrative of the fact that the nebulised droplets are characterised by a polydisperse distribution of size, even if formed by a monodisperse suspension of vesicles. The authors have already indicated the importance of the size of the nebulised droplets with regard to the clinical efficacy of therapeutic aerosols in that only the smallest fraction of particles will succeed in reaching the pulmonary alveoli. The samples analysed exhibit the same distribution of aerodynamic diameter (Table 4). It can be noted that 100% of the population of nebulised droplets has an aerodynamic diameter less than 10 μm. Of this 100%, 99.5% is characterised by an aerodynamic diameter less than 5 μm. 65% of the population with a diameter less than 10 μm has an aerodynamic diameter less than 2 μm and represents the population which will reach the lower airways, that is to say the population useful for carrying beclomethasone dipropionate.

TABLE 4
Aerodynamic diameter values and percentage of the
particles having such aerodynamic diameter

| Aerodynamic diameter (μm) | Percentage |
|---|---|
| <10 | 100 |
| <5 | 99.5 |
| <2 | 65 |

In the study of interaction with bronchial fibroblasts the niosomes containing BDP (BDPE), a drug suspension in the presence of surfactant at the same concentration used in the formation of unstructured niosomes, and the drug mixture on the market were compared. With reference to FIG. 5 it can be noted how the amount of BDP extracted from the bronchial fibroblasts is already greater after the first hour using BDPE and that, over the time interval considered, approximately 80% of the dose administered is found within the cells, this value being much greater than the values obtained with the surfactant-BDP suspension (approximately 10%) and the commercial formulation (approximately 20%). In order to better understand where the drug is released inside the cell, the fibroblasts were incubated with a niosomal suspension containing 6-CF, contained in the aqueous compartment of the vesicles. The cells, having become fluorescent, were analysed by confocal microscopy. Five micrographs (FIG. 6) were obtained after 1, 4, 6, 12 and 24 hours. It can be noted that even after one hour a diffuse fluorescence is evident inside the cellular cytoplasm and increases over time until reaching a maximum after 6 hours. Dotted fluorescence zones can also be seen in this micrograph which represent vesicles not yet broken and probably still present in the endosomes. However, the presence of a diffuse fluorescence is evident in all five micrographs, indicating the release of the vesicular content inside the cytoplasm. The vesicles used therefore are able to carry out intracytoplasmic delivery and therefore may actually be useful for deliverying BDP into the cellular compartment in which the receptor is found. Tests on cellular vitality (MTT test) were carried out from the study of interaction between the non-freeze-dried samples and the cells and these tests demonstrated that the structures used are largely tolerated in that, after 48 hours of incubation, cellular vitality is greater than 90%.

Given the growing interest of pharmaceutical industries in powders which can be administered by inhalation, the authors carried out numerous tests implementing a method which makes it possible to freeze-dry the suspension of the niosomal vesicles containing BDP with the aim of obtaining a freeze-dried product that can be inhaled via dry-powder inhalers. It is known that the process of freeze-drying could break the vesicular structure which, during the process, is subjected to thermal and mechanical stresses, therefore the authors assessed the stability of the vesicles during the process in the early stages of the study. Suspensions of vesicles containing previously dialysed calcein were initially used so as to assess the variation in fluorescence before and after freeze-drying; they were initially freeze-dried both in the absence of cryoprotectant and in the presence of various cryoprotectants: saccharose, lactose, mannitol and trehalose. From the quantitative dose of Tween 20 described above, it was calculated that the percentage of this structured surfactant in the vesicle is equal to 41.5% relative to. the surfactant used in the starting formulation. The ratios by mass of structured Tween 20/cryoprotectant used are 1:0.5, 1:1 and 1:2. The fluorescence results obtained after rehydration of the freeze-dried products were compared with the fluorescence of the vesicular suspension before freeze-drying (‘complete’ vesicles) and with the fluorescence obtained by breaking the bilayer of the vesicles with isopropanol, as illustrated in FIG. 7.

It can be seen from the graphic that better results were obtained using mannitol or trehalose as cryoprotectants, both in a Tween 20/cryoprotectant 1:2 mass ratio.

In order to assess the effective maintenance of the vesicular structure after freeze-drying and resuspension of the freeze-dried product in a buffer, the authors carried out freeze fracture electron microscopy studies (FIG. 8). These studies were carried out both on vesicles containing calcein and on vesicles containing the encapsulated drug. FIG. 8 illustrates the micrographs relating to BDPA, since BDPE demonstrates similar behaviour.

It can be seen from micrograph A, relating to the freeze-dried product resuspended in the absence of cryoprotectant, that the size of the vesicles formed after hydration is greater than those of the original sample (micrograph A′) and that multi-lamellar vesicles are formed from uni-lamellar vesicles. The other micrographs demonstrate the presence of uni-lamellar vesicles that are more or less homogeneous in size; only in the case of the sample freeze-dried in the presence of trehalose with a Tween 20/cryoprotectant 1:2 mass ratio of is there breaking of some vesicles which, following rehydration, form a system of multi vesicular vesicles, that is to say a structure characterised by the presence of more vesicles, small in size, contained in the aqueous compartment of a larger vesicle.

It is clear from these studies and from those regarding fluorescence that the vesicles can be freeze-dried merely in the presence of cryoprotectants and, in particular, that the best results were obtained with mannitol (Tween 20/cryoprotectant 1:2 mass ratio) and trehalose (Tween 20/cryoprotectant 1:1 and 1:2 mass ratio).

Similar results can also be seen in the photos obtained by SEM (FIGS. 9A-B). It is important to note that the powders have a porous nature in all the micrographs; this feature is of key importance for a powder that is to be inhaled; in fact, it is known that the more porous a powder, the more easily it will be able to reach the lower airways. In fact, as a result of their large size and low density, porous particles can be aerosolised more effectively from a DPI (dry-powder inhaler) compared to smaller, non-porous particles, resulting in a greater respirable amount of inhaled drug.

1. A vesicular structure formed of a mixture of a non-ionic surfactant and cholesterol, wherein it is freeze-dried in the presence of a cryoprotectant.

2. The vesicular structure according to claim 1, wherein the non-ionic surfactant is Tween 20.

3. The vesicular structure according to claim 1, wherein the ratio by mass of non-ionic surfactant/cholesterol is 1:1.

4. The vesicular structure according to claim 1, wherein the ratio by mass of non-ionic surfactant/cryoprotectant is between 1:0.5 and 1:2.

5. The vesicular structure according to claim 1, wherein the cryoprotectant is saccharose, lactose, mannitol or trehalose.

6. The vesicular structure according to claim 1, having a diameter between 170 nm and 200 nm.

7. The vesicular structure according to claim 1, wherein said structure has a zeta potential of approximately −40 mV.

8. The vesicular structure according to claim 1, comprising inside a pharmacologically or biologically therapeutic agent.

9-12. (canceled)

13. Method for the preparation of the vesicular structure according to claim 1, comprising:
a) solubilizing a non-ionic surfactant and cholesterol in order to obtain a vesicular structure; and
b) freeze-drying said vesicular structure in the presence of a cryoprotectant.

14. The method according to claim 13, wherein the cryoprotectant is saccharose, lactose, mannitol or trehalose.

15. The method according to claim 13, wherein the ratio by mass of non-ionic surfactant/cryoprotectant is between 1:0.5 and 1:2.

16. The method according to claim 13, wherein freeze-drying is carried out at a pressure of 2 atm. (1520 mm of Hg) and a temperature of −55° C.

17. A pharmaceutical composition comprising the vesicular structure according to claim 1 and suitable solvents and/or diluents.
